(19) United States
(12) Patent Application Publication
(10) Pub. No.: US 2026/0335561 A1
(43) Pub. Date: Oct. 29, 2026

(54) A subassembly for a medicament delivery device

(71) Applicant: SHL Medical AG, Zug (CH)

(72) Inventors: Oscar Alexandersson, Nacka Strand (SE); Johan Zander, Nacka Strand (SE)

(21) Appl. No.: 19/485,308

(22) PCT Filed: May 29, 2024
(86) PCT No.: PCT/EP2024/064760

§ 371 Date: Nov. 18, 2025

(30) Foreign Application Priority Data
Jun. 13, 2023 (EP) 23178852.2

(51) Int. Cl. A61M 5/315

(52) U.S. Cl. CPC A61M 5/31565; A61M 2005/31588; A61M 2205/8206; A61M 2205/8237

(57) ABSTRACT

The present disclosure provides a subassembly for a medicament delivery device accommodating a medicament container. The subassembly includes a housing and an electrical drive unit configured to move a plunger rod in the proximal direction to act on the medicament container. The subassembly further includes a battery configured to power the electrical drive unit. The subassembly further includes a charging port configured to receive a charger having an abutment surface for charging the battery in a charging event. The subassembly further includes an ejector arranged inside the housing adjacent the charging port the ejector having an ejecting surface. The ejector is configured to, during the charging event, be moved into an ejecting state in which the ejecting surface pushes the abutment surface of the charger to thereby eject the charger from the charging port.

### TECHNICAL FIELD

The present disclosure generally relates to medical devices for medicament administration.

### BACKGROUND

A number of medical conditions require injections. These days, a number of different injection devices exist, including various types of pen injectors, autoinjectors and on-body devices. Although many of these devices have enabled major improvements in the management of a number of medical conditions, various limitations do still exist in the current technology.

For example, some medicament delivery devices have an electrical drive unit, such as e.g. a motor, for driving a plunger rod and expelling medicament to the user during a medicament delivery action. Such electrical drive units are typically powered by a battery which periodically is in need of charging. When a charger is charging the battery of the medicament delivery device in a charging event, a simultaneous medicament delivery action may be difficult to achieve as the electrical drive unit may not operate optimally when the battery is charging and/or the medicament delivery device is made more complex due to a more advance electric unit. Thus, there is a need for an efficient ejection of the charger and/or an efficient prevention of charging when performing a medicament delivery action.

In considering these problems, the applicant has appreciated that various developments could be made to help improve the medicament delivery devices on the market today, which are set out in more detail below.

### SUMMARY

An object of the present disclosure is to provide a subassembly for a medicament delivery device, or a medicament delivery device, which solves, or at least mitigates problems of the prior art.

There is hence provided in a first aspect a subassembly for a medicament delivery device accommodating a medicament container, the subassembly comprising: a housing having a proximal end and a distal end; an electrical drive unit configured to move a plunger rod of the medicament delivery device in the proximal direction to act on the medicament container; a battery configured to power the electrical drive unit; a charging port configured to receive a charger having an abutment surface, the charging port having a first electrical connector and being configured to guide a second electrical connector of the charger until the first electrical connector electrically connects to the second electrical connector for charging the battery in a charging event; an ejector arranged inside the housing adjacent the charging port, the ejector having an ejecting surface, wherein the ejector is configured to, during the charging event, be moved into an ejecting state in which the ejecting surface pushes the abutment surface of the charger to thereby eject the charger from the charging port and break the electrical connection between the first and second electrical connectors.

The ejector is thus configured to eject the charger from the charging port by moving into the ejecting state. Hereby, the charging event may be ended, as the electrical connection between the first and second electrical connectors is terminated as the charger is pushed by the ejector as it is moved into the ejecting state. In other words, the ejecting surface of the ejector is arranged and configured to push the abutment surface of the charger as the ejector is moved into the ejecting state. That is, the ejector is configured to physically eject the charger from the charging port by moving into the ejecting state. Thus, there is no need for specific software to be configured to end the charging state. It should be noted that regardless of if the charger is currently charging the battery or not, the ejector is configured to eject the charger from the charging port when being moved into the ejecting state. For example, the battery may be fully charged while the charger is still (physically) arranged in the charging port. For example, the charger may be arranged in a position within the charging port allowing/enabling electrical connection within the charging port, but no charging is occurring as the battery is fully charged. As the charger is ejected from the charging port and the electrical connection between the first and second electrical connectors is terminated or broken, the charger may still be at least partly arranged within the charging port, but in a position preventing the electrical connection between the first and second electrical connectors. The ejecting surface of the ejector may be referred to as a pushing surface. For example, the charging port is a USB charging port and the charger is a USB charger, e.g. any one of USB-A, USB-B, USB-B Mini, USB-B Micro, USB-C.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site.

Correspondingly, when the term “proximal direction” is used, this refers to the direction pointing towards the dose delivery site during use of the medicament delivery device.

When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refers to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component.

Similarly, “radial” or “radially” refer to a direction extending radially relative to the axis, and “rotation”, “rotational” and “rotationally” refer to rotation relative to the axis.

According to one embodiment, the ejector is configured to, in the ejecting state, block the charging port from guiding the second electrical connector of the charger to prevent the first electrical connector from electrically connecting to the second electrical connector. Hereby, when the ejector is arranged in the ejecting state, the charger will not be able to be arranged within the charging port in a position allowing/enabling electrical contact between the first and second electrical connectors. For example, the ejecting surface of the ejector may act as a blocking surface, wherein the blocking surface is arranged to prevent the second electrical connector of the charger to be guided into the charging port to a position allowing/enabling the first electrical connector to electrically connect to the second electrical connector. For example, the ejecting surface or blocking surface may, in ejecting state of the ejector, be arranged to abut the abutment surface of the charger such that the charger cannot move into a position allowing/enabling the first electrical connector to electrically connect to the second electrical connector.

According to one embodiment, the ejector is configured to be moved into the ejecting state by moving relative to the charging port. Thus, the ejector is configured to be moved relative to the charging port in order to eject the charger from the charging port. For example, the ejector is configured to be moved into the ejecting state by being moved close to the charging port then prior to being moved into the ejecting state. However, depending on the arrangement and location of the ejector in the sub-assembly, the opposite may be applicable, i.e. that the ejector is configured to be moved into the ejecting state by being moved closer to the charging port then prior to being moved into the ejecting state.

According to one embodiment, the ejector is configured to be moved into the ejecting state by moving along the charging port. That is, for at least a part of the movement of the ejector into its ejecting state, the ejector is moving along the charging port, e.g. at least partly overlapping with the charging port. In other words, the ejector and the charging port is at least partly overlapping in the axial direction during at least a part of the movement of the ejector into its ejecting state.

According to one embodiment, the distal end of the housing comprises a distally facing surface having an opening, wherein the charging port is arranged inside the housing at a distal end portion of the subassembly and is accessible via the opening. Hereby, the charging port is arranged in an advantageous position within the housing. For example, the charging port is arranged proximally and adjacent to the opening. The opening may be referred to as an access opening for the charging port. The distal end portion of the subassembly may be referred to as a distal part of the subassembly.

According to one embodiment, the ejecting surface is a distally facing surface. Herby, the ejecting surface is particularly suitable for interacting with a charger in the opening in the distally facing surface of the housing. For example, the ejecting surface may have a main extension in a direction that traverse the axial direction of the sub-assembly. However, according to an alternative embodiment, the opening in the housing providing access to the charging port is arranged in an axial surface of the housing, i.e. a surface extending in the proximal or distal direction. In such embodiment, the ejecting surface may still be a distally facing surface, and as the ejector moves into the ejecting state, the charger is squeezed out of the charging port by a transverse movement of the ejector.

According to one embodiment, the ejector is configured to be moved into its ejecting state by being moved distally. Hereby, the ejector is particularly suitable for interacting with a charger in the opening in the distally facing surface of the housing. For example, the ejector is configured to be moved into its ejecting state by being moved distally and the ejecting surface is a distally facing surface. Thus, as the ejector is moved distally, the ejecting surface, being a distally facing surface, a charger being arranged in the charging port via the opening in the distally facing surface of the housing, can be efficiently ejected by that the ejecting surface pushes the abutment surface of the ejector as previously discussed. The abutment surface of the charger may thus be a proximally facing surface when the charger is received in the charging port and the first and second electrical connectors are in electrical contact with each other.

It should be understood that the ejecting surface being a distally facing surface provides an advantageous blocking surface in accordance with the previously mentioned embodiment in which the ejecting surface is arranged to block the charging port from guiding the second electrical connector of the charger to prevent the first electrical connector to electrically connect to the second electrical connector.

According to one embodiment, the ejector is configured to move from: a charging-enabling state in which the ejecting surface is arranged in a non-blocking position enabling the charging port to guide the second electrical connector of the charger such that the electrical connection between the first and second electrical connectors is allowed; into the ejecting state. Hereby, by moving the ejector from the charging enabling state to the ejecting state, the ejector will be ejected from the charging port at least such that the electrical connection between the first and second electrical connectors is broken or terminated. For example, as previously mentioned, the ejector is configured to be moved distally. Thus, in the charging enabling state, the ejector, or at least the ejecting surface, is arranged in a first axial position, and in the ejecting state, the ejector, or at least the ejecting surface, is arranged in a second axial position being distal of the first axial position. Stated differently, in the charging enabling state, the ejecting surface is arranged in a non-pushing interaction with the abutment surface of the charger to thereby enable electrical contact between the first and second electrical connectors. The non-pushing interaction may be achieved by a small gap between the ejecting surface and the abutment surface, or by the ejecting surface is in contact with the abutment surface, but without the ejecting surface subjecting the abutment surface with a pushing force.

According to one embodiment, the charging enabling state is a first state of the ejector and the ejecting state is a second state of the ejector. Thus, the ejector may be configured to be moved from its first state to its second state, in order to eject the charger. The first state may thus occur earlier in time as compared to the second state, with regards to the charging event and ending the charging event.

According to one embodiment, the subassembly further comprises a spring, or flexible arm, configured to bias the ejector to its charging-enabling state. Hereby, the ejector can be moved into its ejecting state by overcoming the biasing force of the spring or flexible arm. For example, the spring or flexible arm is arranged biased in between the housing and the ejector.

According to one embodiment, the subassembly further comprises: a proximal receiving interface adapted to receive a disposable cassette accommodating the medicament container; an activation interface and a first switch, the activation interface being configured to, upon a disposable cassette installation action, being moved by a part of the disposable cassette to actuate the first switch. Thus, the ejector may be configured for a medicament delivery device having a disposable cassette. For example, the housing of the subassembly comprises the proximal receiving interface, typically at a proximal end of the housing. The subassembly may comprise the disposable cassette.

By means of the disposable cassette, variable medicament containers having different volumes are possible to use by subassemblies having the same proximal receiving interface and activation interface. The volume of the medicament container can e.g. range from smaller volumes of about 2 ml to larger volumes of about 10 ml or more depending on the size of the disposable cassette. Regardless of medicament volume, the engagement of the disposable cassette with the proximal receiving interface and activation interface forming the subassembly is the same. The disposable cassette is sometimes simply referred to as “the cassette” throughout this application.

According to one embodiment, the disposable cassette comprises a tubular main body accommodating the medicament container, and at least one distally extending protrusion that extends distally away from a distal end of the tubular main body. The distally extending protrusion may be said part of the disposable cassette that actuates the first switch upon the disposable cassette installation action. Thus, the activation interface may be configured to be pushed in the distal direction by the distally extending protrusion in order to actuate the first switch.

According to one embodiment, the proximal receiving interface comprises a curved distally facing surface adapted to receive and slidably guide a radially extending protrusion of the disposable cassette in a distal direction when the cassette axially rotates relative to the receiving interface until a proximally facing surface of the cassette engages with a distally facing surface of the receiving interface to longitudinally lock the disposable cassette in an attached position in the receiving interface.

According to one embodiment, the activation interface comprises an inclined surface adapted to receive the distally extending protrusion of the cassette when the cassette is further rotated in the receiving interface, whereby an interaction between the distally facing surface of the cassette and the inclined surface of the activation interface cause the activation interface to move distally.

The activation interface may comprise a proximally facing surface adapted to receive the distally extending protrusion of the disposable cassette, wherein the activation interface is configured to, when being pushed distally by the disposable cassette, cause activation of the first switch of the subassembly.

According to one embodiment, the ejector extends from a proximal end to a distal end, wherein the activation interface comprises a distally facing surface configured to, upon the disposable cassette installation action, move to abut against and push the proximal end of the ejector to move the ejector into its ejecting state. Hereby, the ejector is configured to be moved into its ejection state by the activation interface. Thus, the activation interface may be configured to, when being pushed distally by the disposable cassette, cause the ejector to be moved into its ejecting state. Hereby, the subassembly is configured to eject the charger from the charging port upon the disposable cassette installation action. That is, as the disposable cassette is installed into the receiving interface, a charger installed in the charging port will be ejected, preventing charging of the battery when the subassembly has the disposable cassette installed thereto. Hence, a medicament delivery action is prevented while charging.

According to one embodiment, the ejector comprises a tubular portion sized and dimensioned to encompass the second electrical connector, the tubular portion comprising the ejecting surface and being configured to be arranged between the abutment surface of the charger and the charging port during the charging event. Hereby, an efficient arrangement for ejecting the charger as the ejector is moved into its ejecting state is provided. Thus, the ejecting surface may be an annular surface, such as the most distal surface of the tubular portion of the ejector. Correspondingly, the abutment surface of the ejector may be an annular surface, such as the annular surface surrounding the second electrical connector.

According to one embodiment, the subassembly further comprises an electric motor configured to move the ejector into its ejecting state in response to the actuation of the first switch. Hereby, the ejector is configured to be moved into its ejection state by the electric motor. Thus, the electric motor may be configured to, when the first switch is actuated, cause the ejector to be moved into its ejecting state. Hereby, the subassembly is configured to eject the charger from the charging port upon the disposable cassette installation action. That is, as the disposable cassette is installed into the receiving interface and the activating interface moves to actuate the first switch, a charger installed in the charging port will be ejected, preventing charging of the battery when the subassembly has the disposable cassette installed thereto. Hence, a medicament delivery action is prevented while charging.

According to one embodiment, the electric motor is a linear motor having a movable piston connected to, or comprising, the ejector. That is, the movable piston is moved upon actuation of the first switch, whereby the piston is arranged to act upon the ejector such that the ejector is moved into its ejecting state. As mentioned, the movable piston may comprise the ejector, such that an end portion of the movable piston comprises the ejecting surface.

According to one embodiment, the subassembly further comprises a second switch, wherein the activation interface comprises a first distally extending arm extending towards the first switch and a second distally extending arm extending towards the second switch, and wherein the first distally extending arm is configured to, upon a first movement of the activation interface in the distal direction, reach and actuate the first switch, and wherein the second distally extending arm is configured to, upon a second movement of the activation interface in the distal direction, reach and actuate the second switch. For example, the actuation of the first switch results in the ejector being moved into its ejecting state as previously described, and the actuation of the second switch actuates a medicament delivery action (or event).

A medicament delivery action or event may e.g. be achieved by the plunger rod is moved proximally by the electrical drive unit powered by the battery to act on the medicament container.

According to one embodiment, and with reference to the previously mentioned embodiment including the described interaction between the distally facing surface of the cassette and the inclined surface of the activation interface causing the activation interface to move distally, such distal motion of the activation interface causes the second arm to reach and active the second switch. According to one embodiment, the distal ends of the first arm and the second arm are asymmetric.

According to one embodiment, the subassembly comprises an activation spring arranged to spring load the activation interface away from the first switch and the second switch.

According to a second aspect of the present disclosure, there is provided with a subassembly for a medicament delivery device comprising a disposable cassette accommodating a medicament container. The subassembly comprises: a housing having a proximal end and a distal end; a proximal receiving interface adapted to receive the disposable cassette; an activation interface and a first switch, the activation interface being configured to, upon a disposable cassette installation action, being moved by a part of the disposable cassette to actuate the first switch; an electrical drive unit configured to move a plunger rod of the medicament delivery device in the proximal direction to act on the medicament container; a battery configured to power the electrical drive unit; a charging port configured to receive a charger having an abutment surface, the charging port having a first electrical connector and being configured to guide a second electrical connector of the charger until the first electrical connector electrically connects to the second electrical connector for charging the battery in a charging event; an electric motor configured to, during the charging event, act on the activation interface to prevent the activation interface to move in the distal direction and thereby preventing actuation of the first switch.

The effects and features of the second aspect are largely analogous to those described above in connection with the first aspect. Embodiments mentioned in relation to the first aspect, in particular those of the receiving interface, the activation interface, the first switch, as well as the second switch, are entirely, or largely, compatible with the second aspect, and vice versa, of which some are exemplified below.

Thus, according to the second aspect, the disposable cassette is prevented from being installed into the receiving interface and the activating interface is thus prevented from being moved to actuate the first switch, when the electric motor is arranged to act on the activation interface to prevent the activation interface to move in the distal direction. Hereby, charging of the battery is allowed as long as the disposable cassette is not installed into the receiving recess. Hence, a medicament delivery action is prevented while charging.

According to one embodiment, the subassembly further comprises a blocking arm configured to be moved by the electric motor, wherein the blocking arm is arranged to abut the activation interface during the charging event to prevent the activation interface to move in the distal direction. Hereby, an efficient structure for preventing the activation interface to move in the distal direction is provided. As described in the first aspect, the electric motor may be a linear motor. The linear motor of the second aspect may comprise a movable piston connected to, or comprising, the blocking arm. That is, the movable piston is arranged to move into a position to act upon the blocking arm such that the blocking arm is arranged to abut the activation interface. As mentioned, the movable piston may comprise the blocking arm, such that an end portion of the movable piston comprises the blocking arm.

According to one embodiment, the electric motor is configured to, upon ceasing of the charging event and removal of the charger from the charging port, move the blocking arm distant from the activation interface to enable the activation interface to move in the distal direction and actuate the first switch. Hereby, the blocking arm is configured to be moved distant from the activation interface, e.g. in response to that the electric motor receives a signal indicating that the charging event is stopped and/or that the charger has been ejecting from the charging port. That is, as the disposable cassette is installed into the receiving interface, the blocking arm has been moved away from the activation interface ins response to and the charger is no longer installed in the charging port, preventing charging of the battery when the subassembly has the disposable cassette installed thereto.

According to a third aspect of the present disclosure, there is provided a medicament delivery device comprising the subassembly according to the first aspect or second aspect of the present disclosure.

Effects and features of the third aspect are largely analogous to those described above in connection with the first aspect and second aspect. Embodiments mentioned in relation to the first aspect and second aspect are largely compatible with the third aspect, and vice versa.

According to one embodiment, applicable to any one of the first to third aspects of the disclosure, the subassembly comprises a control unit being configured to identify the installation of the disposable cassette into the proximal receiving interface. Such control unit may e.g. be configured to determine whether the charger is installed into the charging port or not, and be configured to transmit a signal indicating whether the charger is installed into the charging port or not.

It should be understood that the charger is typically not included in the sub-assembly and/or the medicament delivery device. However, according to at least one example embodiment, the sub-assembly comprises the charger, the charger is configured to be removably arranged relative to the charging port. The charger is typically a charger having an abutment surface arranged adjacent to the second electrical contact. The charger may e.g. comprise a flange from which the second electrical contact extends, wherein the flange comprises the abutment surface. The abutment surface is typically facing in the direction of the second electrical contact. In other words, the charging port is configured to receive a charger having such a flange and abutment surface. The second electrical connector of the charger is typically electrically connected to an external power supply, e.g. the power grid or an external battery pack.

According to one embodiment, applicable to any one of the first to third aspects of the disclosure, the disposable cassette comprises an electronic tag attached to a distal end of the tubular main body, the electronic tag having stored data that is related to the medicament contained within the tubular main body, wherein the data is readable by an electronic reader of the subassembly or medicament delivery device when the disposable cassette is in the attached position. The electronic tag advantageously enables for providing the data to the medicament delivery device to make corresponding settings. The stored data may furthermore be related to the volume of the medicament container.

According to one embodiment, applicable to any one of the first to third aspects of the disclosure, the previously mentioned actuation of the first switch activates the electronic reader. Thus, when the disposable cassette is fully inserted in the receiving interface, or at least sufficiently to reach the activation interface, the activation interface is advantageous pushed without any additional required action from the user, to activate the electronic reader.

According to one embodiment, applicable to any one of the first to third aspects of the disclosure, the medicament container is a syringe. The medicament delivery member may thus be a needle. According to one embodiment, the medicament delivery device comprises an infusion set including a flexible tube connected to a through hole of the disposable cassette at the proximal end thereof. At the other end of the tube, a needle is typically attached via a pad at a medicament delivery site.

Generally, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 is a perspective, partly exploded, view of a medicament delivery device according to embodiments of the present disclosure;

FIG. 2 is a perspective, partly exploded view of a subassembly of the medicament delivery device according to embodiments of the present disclosure;

FIGS. 3A-3B are perspective views of parts of the subassembly of FIG. 2 according to embodiments of the present disclosure;

FIGS. 4A-4B are close-up perspective views of the interface between a charger and a charging port of the subassembly of FIG. 2 according to embodiments of the present disclosure;

FIG. 5 is a close-up perspective view of a distal end of a disposable cassette and an activation interface according to embodiments of the present disclosure;

FIG. 6 is a close-up perspective view of a distal end of the disposable cassette and the activation interface according to embodiments of the present disclosure;

FIGS. 7A-7B are perspective, partly schematic, views of another subassembly according to embodiments of the present disclosure;

FIGS. 8A-8B are perspective, partly schematic, views; of another subassembly according to embodiments of the present disclosure.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

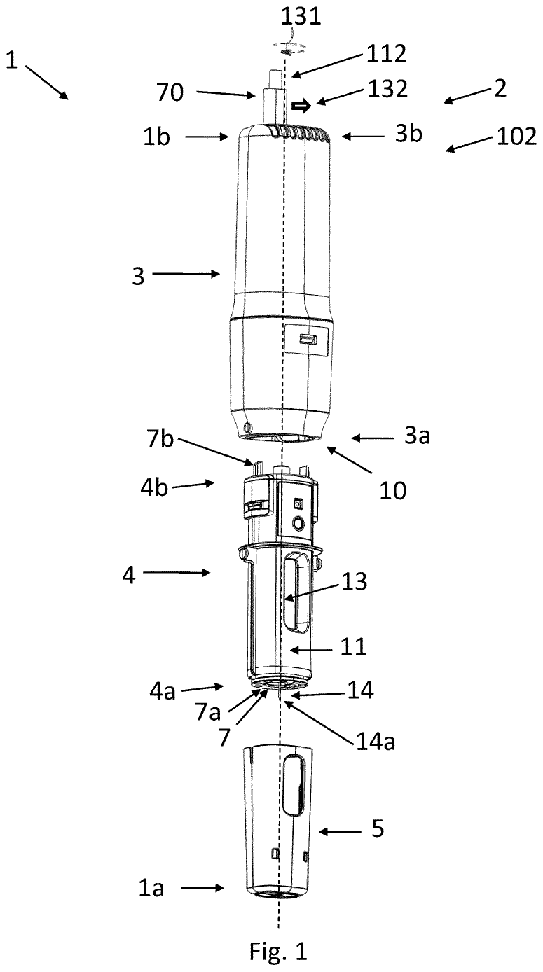

FIG. 1 shows an example of a medicament delivery device 1 such as an autoinjector of an injection device according to embodiments of the present disclosure. The medicament delivery device 1 is configured to expel medicament from a medicament container 13 via a medicament delivery member 14, here embodied as a needle, to a user at a dose delivery site. The medicament delivery device 1 extends from a proximal end 1a to a distal end 1b relative to the axis 112.

The axis 112 is in FIG. 1 a centre axis, from which a circumferential direction 131 relative to the centre axis 112 and a radial direction 132 extending radially relative to the centre axis 112, can be defined.

The medicament delivery device 1 comprises a subassembly 2 which is connectable to a disposable cassette 4 having a proximal end 4a and a distal end 4b. The subassembly 2 comprises a housing 3 having a corresponding proximal end 3a and a distal end 3b. The housing 3, and its internal components and excluding the disposable cassette 4 may be referred to as a power module. According to such embodiment, the power module is connectable to the disposable cassette 4, and the subassembly 2 may be referring to the power module and/or the disposable cassette 4.

The disposable cassette 4 comprises a tubular main body 11 accommodating the medicament container 13 and the thereto arranged medicament delivery member 14 having a proximal end 14a. In the embodiment of FIG. 1, in which the medicament delivery member 14 is a needle, the proximal end 14a is the tip of the needle. The disposable cassette 4 is typically adapted to accommodate medicament containers of different volumes, for example ranging from about 1 ml to about 10 ml.

The housing 3 comprises a receiving interface 10 configured to received and lock the disposable cassette 4 to the proximal end 3a of the housing 3.

The disposable cassette 4 comprises a cap 5 arranged to protect the medicament delivery member 14. In FIG. 1, the cap 5 is shown proximally of the disposable cassette 4, after the cap 5 has been removed from the medicament delivery member 14 during a cap removal action.

The disposable cassette 4 may comprise a delivery member cover 7 configured to cover the delivery member 14. The delivery member cover 7 is configured to move axially relative to the tubular main body 11. For example, the delivery member cover 7 may be moved from a protracted position into a retracted position, shown in FIG. 1, in which the delivery member cover 7 is received further into the tubular main body 11 in order to expose the delivery member 14 of the medicament container 13.

In FIG. 1, an external charger 70 is connected to a charging port of the subassembly 2, as will be described in further detail in the following.

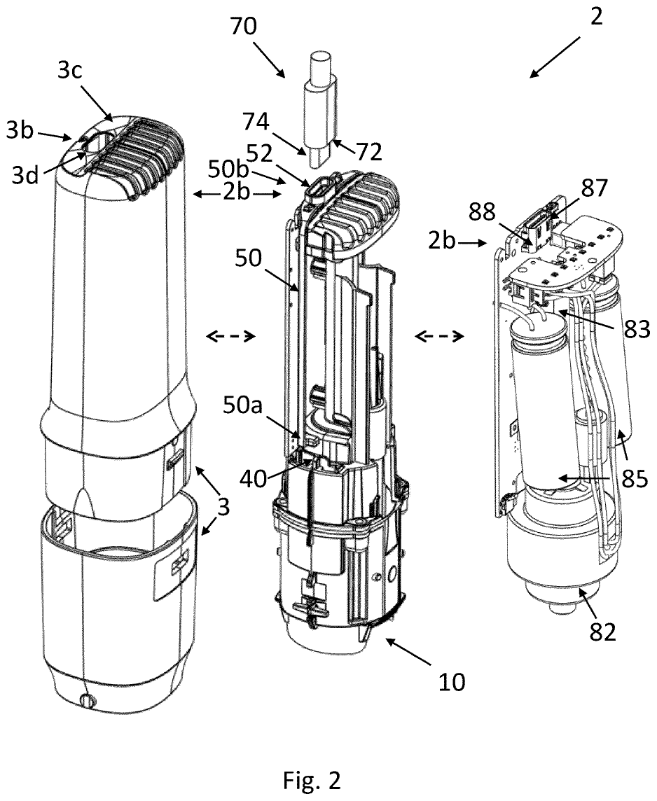

FIG. 2 is a partly exploded view of the medicament delivery device 1 with the housing 3 removed from the subassembly 2 to show the internal components of the housing 3. As shown in FIG. 2, the housing 3 may be divided into two parts, a first housing part and a second housing part, wherein the second housing part is arranged distally of the first housing part.

The subassembly 2 comprises a plunger rod 83 configured to move proximally to act on the medicament container 13 to expel medicament via the proximal end 14a of the medicament delivery member 14. The subassembly 2 further comprises an electrical drive unit 82 configured to move the plunger rod 83 in the proximal direction. The electrical drive unit 82 may e.g. be an electrical motor.

The subassembly 2 further comprises a battery 85 configured to power the electrical drive unit 82. That is, by the electric energy of the battery 85, the electrical drive unit 82 is capable of moving the plunger rod 83 in the proximal direction to act on the medicament container 13.

A charging port 87 is arranged in a proximal end portion 2b of the subassembly 2. The charging port 87 is configured to receive a charger 70 having an abutment surface 72 and a second electrical connector 74. The charging port 87 comprises a first electrical connector 88 and is configured to guide the second electrical connector 74 of the charger 70 until the first electrical connector 88 electrically connects to the second electrical connector 74. Typically, the first electrical connector 88 is then in direct contact with the second electrical connector 74. Hereby, the charger 70 may charge the battery 85 in a charging event. Thus, the charging port 87 is electrically connected to the battery 85. In more detail, in the embodiment of FIG. 2, the distal end 3b of the housing 3 comprises a distally facing surface 3c having an opening 3d. The charging port 87 is arranged inside the housing 3 at the distal end portion 2b thereof and is accessible via the opening 3d. Thus, the charger 70 is installed into the charging port 87 that the second electrical connector 74 is moved through the opening 3d and into the charging port 87.

As shown in FIG. 2, the subassembly 2 comprises an ejector 50 arranged inside the housing 3 adjacent the charging port 87. The ejector extends from a proximal end 50a to a distal end 50b and comprises an ejecting surface 52 arranged in the distal end 50b.

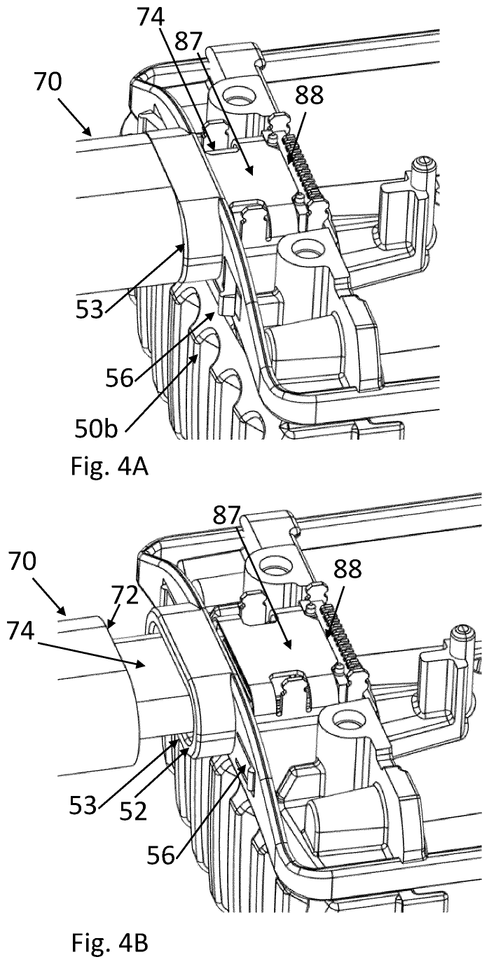

The ejector 50 is configured to, during the charging event, be moved into an ejecting state in which the ejecting surface 52 pushes the abutment surface 72 of the charger 70 to thereby eject the charger 70 from the charging port 87, and break the electrical connection between the first and second electrical connectors 88, 74, as will be described in further detail with reference to FIGS. 4A-4B.

As mentioned with reference to FIG. 1, the subassembly 2 comprises a proximal receiving interface 10 adapted to receive a disposable cassette 4 accommodating the medicament container 13. Moreover, the subassembly 2 of the embodiment of FIG. 2 comprises an activation interface 40 configured to, upon a disposable cassette installation action, be moved distally by a part of the disposable cassette 4.

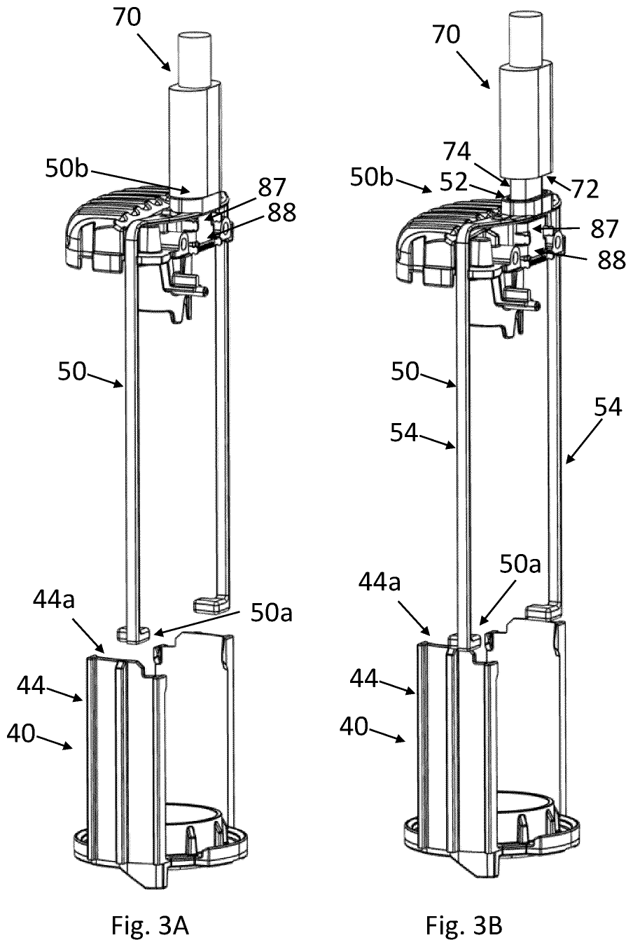

Turning to FIGS. 3A-3B, showing the activation interface 40, the ejector 50, the charging port 87 and the charger 70 of FIG. 2. The activation interface 40 comprises a distally facing surface 44a configured to, upon the disposable cassette installation action, move to abut against and push the proximal end 50a of the ejector 50 to move the ejector 50 into its ejecting state, shown in FIG. 3B. That is, as the activation interface 40 is moved distally from the state shown in FIG. 3A, by the installation of the disposable cassette 4 into the receiving interface 10, the distally facing surface 44a pushes the ejector 50 in the distal direction by interacting with the proximal end 50a of the ejector 50. As the ejector 50 is moved in the distal direction, the distal end 50b of the ejector comprising the ejecting surface is moved distally and thus pushes the abutment surface 72 of the charger 70 to eject the charger 70 from the charging port 87.

In more detail, the ejector 50 is configured to move from a charging-enabling state shown in FIG. 3A, in which the ejecting surface 52 of the ejector 50 is arranged in a non-blocking position enabling the charger 70 to be received in the charging port 87 such that the electrical connection between the first and second electrical connectors 88, 74 is allowed, into the ejecting state shown in FIG. 3B in which the ejecting surface 52 pushes the abutment surface 72 of the charger 70 to thereby eject the charger 70 from the charging port 87. In other words, the activation interface 40 moves from a first axial position in the charging enabling state to a second axial position in the ejecting state (owing to the disposable cassette installation action), wherein the second axial position is distal of the first axial position.

Turning to FIGS. 4A-4B showing enlarged views of the interface between the charger 70 and the charging port 87 in the charging enabling state (FIG. 4A) and the ejecting state (FIG. 4B). In the charging enabling state in FIG. 4A, the charger 70 is installed in the charging port 87 and the first electrical connector 88 electrically connects to the second electrical connector 74 for charging the battery 85. In the ejecting state in FIG. 4B, as a result of the previously described distal movement of the ejector 50, the ejecting surface 52 pushes the abutment surface 72 of the charger 70 to thereby eject the charger 70 from the charging port 87, and breaks the electrical connection between the first and second electrical connectors 88, 74.

As shown in FIG. 4A, the ejector 50 may comprise a tubular portion 53 sized and dimensioned to encompass the second electrical connector 74. The tubular portion 53 may comprise the ejecting surface 52 and be arranged between the abutment surface 72 of the charger 70 and the charging port 87 during the charging event, as shown in FIG. 4A. Thus, the ejecting surface 52 may be an annular surface of the tubular portion 53. Correspondingly, the abutment surface 72 of the ejector 70 is typically an annular surface, such as the annular surface surrounding the second electrical connector 74. Hereby, the ejection of the ejector 70 may be improved.

Moreover, in the ejecting state shown in FIG. 4B, the ejector 50 is configured to, in the ejecting state, block the charging port 87 from guiding the second electrical connector 74 of the charger 70. Hereby, the first electrical connector 88 is prevented from electrically connecting to the second electrical connector 74. In other words, as the ejector 50 has been moved distally into the ejecting state, the ejecting surface 52 will abut the abutment surface 72 of the charger 72 at a position in which the second electrical connector 74 cannot be in electrical contact with the first electrical connector 88 of the charging port 87.

In the embodiment of FIGS. 3A-3B and 4A-4B, the ejector 50 is configured to be moved into the ejecting state by moving relative to the charging port 87. That is, the ejector 50, and the ejecting surface 52, is moved relative to the charging port 87 when moving from the charging enabling state to the ejecting state. In the embodiment of FIGS. 3A-3B and 4A-4B, the ejector 50 is moved at least partly along the charging port 87. For example, in the charging enabling state shown in FIG. 4A, the tubular portion 53 of the ejector 50 at least partly overlaps with the charging port 87 in the axial direction, and in the ejecting state shown in FIG. 4B, the tubular portion 53 of the ejector 50 has moved distally in the axial direction to no longer overlap with the charging port 87.

As mentioned with reference to FIG. 2, the charging port 87 is arranged inside the housing 3 at the distal end portion 2b and is accessible via the opening 3d in the distally facing surface 3c. Thus, as the charger 70 is installed into the charging port 87 via the distally facing surface 3c, the ejector 50 is configured to be moved into its ejecting state by being moved distally, and the ejecting surface 52 is a distally facing surface, as shown in FIGS. 4A-4B.

The subassembly 2 may comprise a spring, or flexible arm 56, configured to bias the ejector 50 to its charging enabling state. In the embodiment shown in FIGS. 4A-4B, the flexible arm 56 is arranged biased in between an inner surface of the housing 3 (shown in FIG. 2) and the distal end 50b of the ejector 50. The flexible arm 56 may be integrated into the ejector 50, such as the distal end 50b of the ejector. The ejector 50 can thus be moved into its ejecting state by overcoming the biasing force of the flexible arm 56.

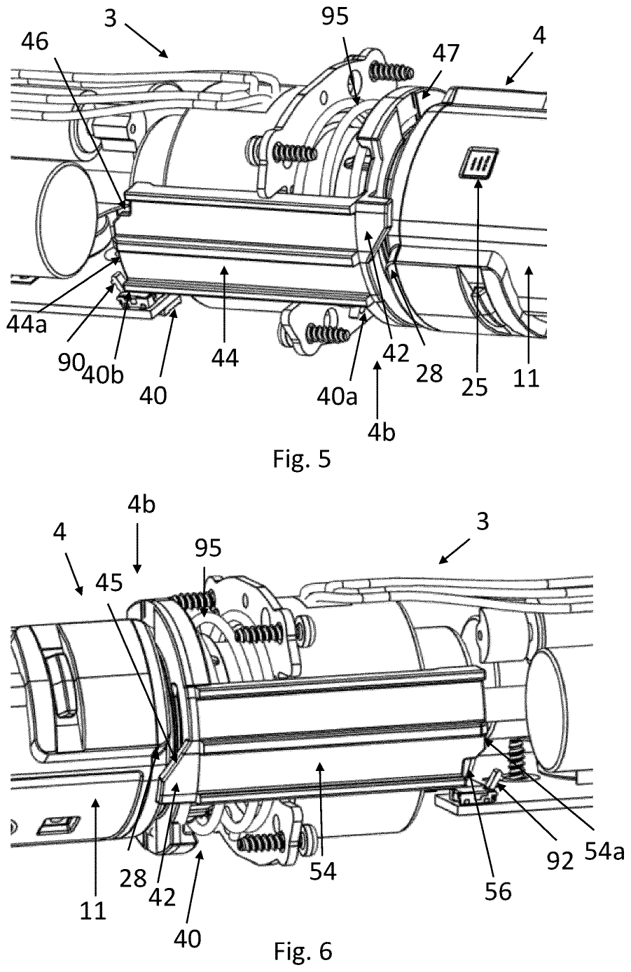

The disposable cassette installation action will now be further described with reference to FIGS. 5 and 6. FIGS. 5 and 6 are close-up views of the distal end 4b of the disposable cassette 4 inserted in the receiving interface 10 but not yet mounted by rotating the disposable cassette 4 in the receiving surface 10.

The activation interface 40 comprises a proximal end 40a and a distal end 40b. The proximal end 40a comprises protrusions 42 that extend in a proximal direction. The protrusions 42 are configured to receive distally extending protrusions 28 of the cassette 4 when the cassette 4 is rotated as part of the disposable cassette installation action. Each one of the protrusions 42 comprises an inclined surface 45 adapted to receive a distally facing inclined surface of the distally extending protrusions 28 of the disposable cassette 4 when the disposable cassette 4 is rotated.

The activation interface 40 comprises a first distally extending arm 44 (shown in FIG. 5) and a second distally extending arm 54 (shown in FIG. 6). Each one of the first and second distally extending arms 44, 54 comprises a cut-out 46, 56 at the distal end 40b of the activation interface 40, the cut-outs 46, 56 being arranged diagonally with respect to each other so that the first and second distally extending arm 44, 54 are asymmetric.

The activation interface 10 may further comprise longitudinal ribs on the respective first and second distally extending arms 44, 54, that are configured to fit in and slide in corresponding slots or grooves on a radially inwards facing surface of the receiving interface 10.

In FIG. 5, the first distally extending arm 44 of the activation interface 40 extends towards a first switch 90. On the opposite side, as is shown in FIG. 6, the second distally extending arm 54 extends towards a second switch 92. Furthermore, a spring 95 is arranged to spring load the activation interface 40 away from the first switch 90 and the second switch 92.

As the disposable cassette 4 is inserted in the receiving interface 10 and mounted by rotating the disposable cassette 4 in the receiving surface 10, during an axial rotation of the disposable cassette 4, a radially extending protrusion slides on the curved distally facing surface of the receiving interface 10. This sliding motion on the curved surface causes a distal axial motion of the disposable cassette 4 so that the distally facing surface of the protrusions 28 abut against and push on a proximally facing surface 47 the activation interface 40 in a distal direction. In more detail, the distally facing inclined surfaces of the protrusions 28 interact with the inclined surfaces 45 of the protrusions 42 of the activation interface 40 when the disposable cassette 4 is rotated. The axial rotation for locking of disposable cassette 4 may be about 60 degrees with respect to the receiving interface 10. The spring 95 is compressed by the axial motion of the activation interface 40 caused by the force from the disposable cassette 4 moving distally when mounted in the receiving interface 10.

Thus, upon axial rotation of the disposable cassette 4, the first distally extending arm 44 of the activation interface 40 will activate the first switch 90. As the cut-out 46 of the first distally extending arm 44 is asymmetrically arranged with respect to the cut-out 56 of the second distally extending arm 54 (as shown by comparing FIGS. 5 and 6), in the same position of the activation interface 40 in the receiving interface 10, the second distally extending arm 54 of the activation interface 40 does not reach to activate the second switch 92. Thus, the second switch 92 is not activated by mounting the disposable cassette 4 in the receiving interface 10.

The second switch 92 may be activated by moving the delivery member cover 7 (shown in FIG. 1). Typically, upon exertion of a force of the delivery member cover 7 in the distal direction, e.g. by pushing a proximal end za of the delivery member cover 7 against a medicament delivery site, a distal end 7b of the delivery member cover 7 interacts with the activation interface 40 causing the activation interface 40 to move further distally such that the second distally extending arm 54 reach to activate the second switch 92.

Thus, the first distally extending arm 44 is configured to, upon a first movement of the activation interface 40 in the distal direction, reach and actuate the first switch 90, and the second distally extending arm 54 is configured to, upon a second movement of the activation interface 40 in the distal direction, reach and actuate the second switch 92.

A rotation of the disposable cassette 4 in the opposite direction causes the activation interface 40 to move proximally by the force applied by the spring 95. This may cause the deactivation of the first switch 90 and/or the second switch 92 depending on the degree of rotation of the disposable cassette 4 that allows the activation interface 40 to move variable distances in the proximal direction. A further rotation in this opposite direction allows for dismounting the cassette 4 from the receiving interface 10 so that it can be disposed of. Subsequently, a new cassette 4 can be mounted to the receiving interface 10 as described herein.

Activation of the first switch 90 may e.g. cause activation of an electronic reader of the subassembly 2 to read an electronic tag 25 of the cassette 4. Activation of the second switch 92 may e.g. actuate movement of the plunger rod 83 in the proximal direction to act on the medicament container 13 to expel medicament through the proximal end 14a of the medicament delivery member 14. As previously described, the plunger rod 83 is moved by the electrical drive unit 82 powered by the battery 85. The disposable cassette 4 may comprise a plunger rod adapter fitted in the tubular main body 11. The plunger rod adapter is preferably cylindrical and is adapted to be pushed proximally by the plunger rod 83 during a medicament delivery action. The proximal motion of the plunger rod adapter cause the expulsion of a medicament from the medicament container 13.

Thus, as described for FIGS. 3A-3B, upon the disposable cassette installation action, the activation interface 40 and the distally facing surface 44a of the first distally extending arm 44 moves to abut against and push the proximal end 50a of the ejector 50 to move the ejector 50 into its ejecting state.

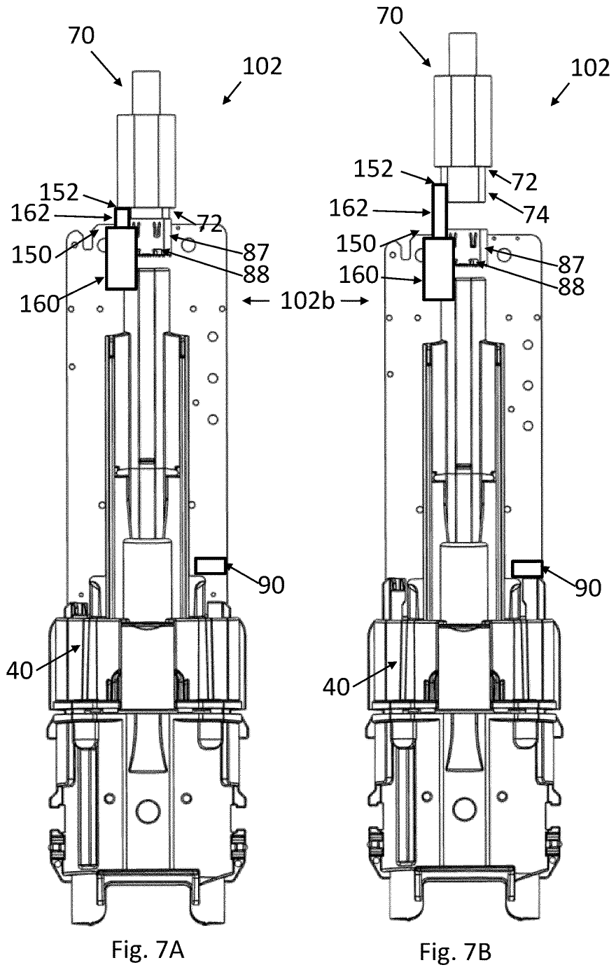

FIGS. 7A-7B discloses partly schematic views of a subassembly 102 according to another embodiment of the present disclosure. The subassembly 102 may be identical to the embodiments of the subassembly 2 previously described, with the exception of the ejector. For example, the same housing 3 with internal components, the charging port 87, the first electrical connector 88, the proximal receiving interface 10, the activation interface 40, the first and second switches 90, 92 and the interaction with the disposable cassette 4 may be the same for subassembly 102. Therefore, only the ejector 150 will be described in detail with reference to FIGS. 7A-7B.

In FIGS. 7A-7B, the housing 3 is removed for increased visibility. In FIG. 7A, the charger 70 is installed into the charging port 87 such that the first electrical connector 88 electrically connects to the second electrical connector 74 for charging the battery 85 in a charging event as previously described. Thus, the ejector 150 is in FIG. 7A arranged in its charging enabling state.

The subassembly 102 comprises electric motor 160 arranged at a distal end portion 102b of the subassembly 102. The electric motor 160 is a linear motor having a movable piston 162. As shown in FIG. 7A, the movable piston 160 extends axially in the distal direction towards the charger 70. The ejector 150 in the subassembly 102 is at least partly comprised of the electric motor, as the ejecting surface 152 is formed as the distal end of the movable piston 162. The ejecting surface 152 is thus a distally facing surface.

As the electric motor 160 is operated, the movable piston 162 is moved distally, and the ejecting surface 152 abuts the abutments surface 72 of the charger 70 to eject the charger 70 from the charging port 87. Thus, correspondingly to the subassembly 2, the ejector 150 is configured to, during the charging event, be moved into the ejecting state in which the ejecting surface 152 pushes the abutment surface 72 of the charger 70 thereby ejecting the charger 70 from the charging port 87 and break the electrical connection between the first and second electrical connectors 88, 74.

The electric motor 160 may e.g. be configured to move the ejector 150 into its ejecting state in response to the actuation of the first switch 90, as described with reference to FIG. 5. Thus, the electric motor 160 is configured to, when the first switch 90 is actuated, cause the ejector 150 to be moved into is the ejecting state, shown in FIG. 7B. Hereby, the subassembly 102 is configured to eject the charger 70 from the charging port 87 upon the disposable cassette installation action. That is, as the disposable cassette 4 is installed into the receiving interface 10 and the activating interface 40 moves to actuate the first switch 90, the charger 70 installed in the charging port 87 will be ejected, preventing charging of the battery 85 when the subassembly 102 has the disposable cassette 4 installed thereto.

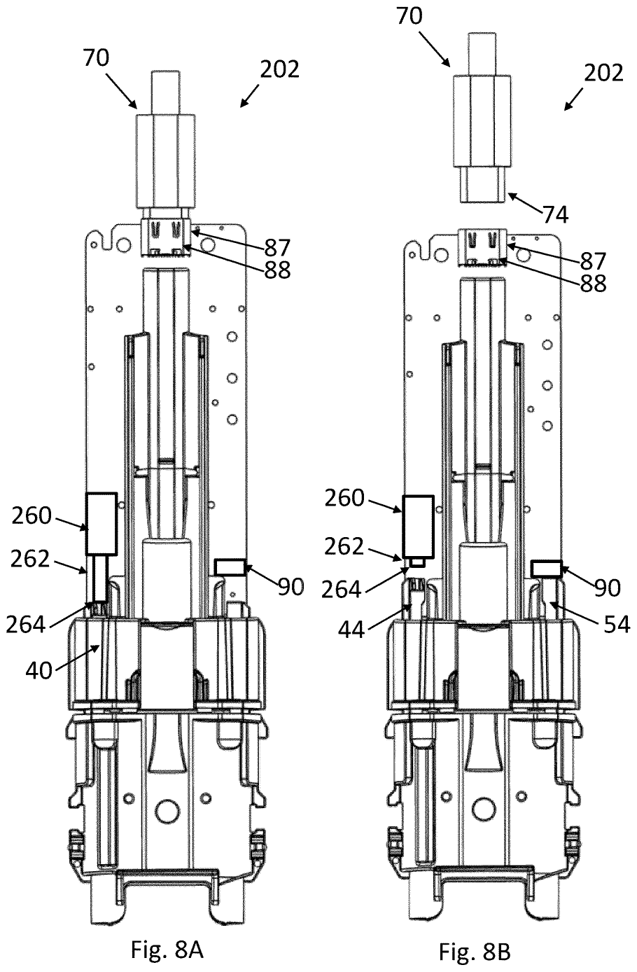

FIGS. 8A-8B discloses partly schematic views of a subassembly 202 according to another embodiment of the present disclosure. The subassembly 202 may be identical to the embodiments of the subassembly 2 previously described, with the exception of the ejector. For example, the same housing 3 with internal components, the charging port 87, the first electrical connector 88, the proximal receiving interface 10, the activation interface 40, the first and second switches 90, 92 and the interaction with the disposable cassette 4 may be the same for subassembly 202. The subassembly 202 may be comprised of the medicament delivery device 1, shown in FIG. 1. The subassembly 202 is thus connectable to the disposable cassette 4 in a corresponding manner as previously described.

In more detail, and with reference to FIGS. 1-2, the subassembly 202 comprises the housing 3 with the proximal end 3a and the distal end 3b, the receiving interface 10 configured to receive and lock the disposable cassette 4 to the proximal end 3a of the housing 3, the activation interface 40 and at least the first switch 90 described with reference to FIGS. 5-6. Moreover, the subassembly 202 comprises the electrical drive unit 82 configured to move the plunger rod 83 of the medicament delivery device 1 in the proximal direction to act on the medicament container 13, the battery 85 configured to power the electrical drive unit 82, the charging port 87 configured to receive the charger 70 having the abutment surface 72. The charging port 87 has a first electrical connector 88 and is configured to guide the second electrical connector 74 of the charger 70 until the first electrical connector 88 electrically connects to the second electrical connector 74 for charging the battery 83 in a charging event, as previously described.

The subassembly 202 further comprises an electric motor 260 configured to, during the charging event, act on the activation interface 40 to prevent the activation interface 40 to move in the distal direction and thereby preventing actuation of the first switch 90. In more detail, the electric motor 260, e.g. a linear motor, comprises a blocking arm 262 configured to be moved by the electric motor 260. The blocking arm 262 may comprise a proximally facing surface 264 configured to abut the activation interface 40, such as e.g. the distally facing surface 44a of the first distally extending arm 44, during the charging event to prevent the activation interface 40 to move in the distal direction, and the first distally extending arm 44 to reach the first switch 90.

Thus, the disposable cassette 4 is prevented from being installed into the receiving interface 10 and the activating interface 40 is thus prevented from being moved to actuate the first switch 90 when the electric motor is arranged to act on the activation interface 40 to prevent the activation interface 40 to move in the distal direction. Hereby, charging of the battery 85 is allowed as long as the disposable cassette 4 is not installed into the receiving recess 10 of the subassembly 202.

The electric motor 260 may be configured to, upon ceasing of the charging event and removal of the charger 70 from the charging port 87, as shown in FIG. 8B, move the blocking arm 262 and the proximally facing surface 264 distant from the activation interface 40, such as being distant from the distally facing surface 44a of the first distally extending arm 44, to enable the activation interface 40 to move in the distal direction and actuating the first switch 90. Hereby, the blocking arm 262 is configured to be moved distant from the activation interface 40, e.g. in response to that the electric motor 260 receives a signal indicating that the charging event is stopped and/or that the charger 70 has been ejecting from the charging port 87. That is, as the disposable cassette 4 is installed into the receiving interface 10 of the subassembly 202, the blocking arm 262 has been moved away from the activation interface 40 in response to that the charger 70 is no longer installed in the charging port 87, preventing charging of the battery 85 when the subassembly 202 has the disposable cassette 4 installed thereto.

As a further alternative embodiment, the electric motor 260 may be configured to, during the charging event, act on the activation interface 40 to prevent the activation interface 40 to move in the distal direction and thereby preventing actuation of the second switch 92 Thus, the blocking arm 262 may be configured to abut the activation interface 40, such as e.g. a distally facing surface of the second distally extending arm 54, during the charging event to prevent the activation interface 40 to move in the distal direction, and the second distally extending arm 54 to reach the second switch 92.

It should be noted that instead of using the first and second electrical connectors 88, 74 being in direct contact with each other for charging the battery 85, the charger and the charging port may be configured for wireless charging. To avoid simultaneous charging of the battery 85 and a medicament delivery action, e.g. in response to activating the second switch 92 as previously described, the controller of the medicament delivery device may be configured to detect a wireless charging connection, and in response to such detection, disabled the electrical drive unit 82 so the electrical drive unit 82 cannot actuate movement of the plunger rod 83 in the proximal direction.

As a further alternative to avoid simultaneous charging of the battery 85 and a medicament delivery action, an activation sensor, such as e.g. the second switch 92, may be electrically disabled or blocked so that a controller of the medicament delivery device cannot receive the activation data from the activation sensor when the charger is charging the battery 85.

As a further alternative to avoid simultaneous charging of the battery 85 and a medicament delivery action, the charging port and the first electrical connector may be arranged inside the housing in a position which is occupied by the disposable cassette when the latter is installed in the receiving interface. Thus, the charger cannot be installed into the charging port at the same time as the disposable cassette is installed in the receiving interface.

The medicament delivery devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjögren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behçet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the medicament containers, and administrated by the medicament delivery devices described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the medicament containers, and administrated by the medicament delivery devices described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the medicament containers, and administrated by the medicament delivery devices described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C-C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin gene-related peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, B-lymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation w137 (CDW137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDw123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies.

Exemplary drugs that could be included in the medicament containers, and administrated by the medicament delivery devices described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-1a, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the medicament containers, and administrated by the medicament delivery devices described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the medicament containers, and administrated by the medicament delivery devices described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the medicament containers, and administrated by the medicament delivery devices described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the medicament containers, and administrated by the medicament delivery devices described herein also include, but are not limited to, analgesics (e.g., acetaminophen), antipyretics, corticosteroids (e.g. hydrocortisone, dexamethasone, or methylprednisolone), antihistamines (e.g., diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the medicament containers, and administrated by the medicament delivery devices described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g. an animal-derived, human-derived, or recombinant hyaluronidase enzyme), concentration modifiers or enhancers, stabilizers, buffers, or other excipients.

Exemplary drugs that could be included in the medicament containers, and administrated by the medicament delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

The inventive concept has mainly been described above with reference to a few examples. However, as is readily appreciated by a person skilled in the art, other embodiments than the ones disclosed above are equally possible within the scope of the inventive concept, as defined by the appended claims.

Some other aspects of the invention are disclosed in the following clauses.
1. A subassembly (2, 102) for a medicament delivery device (1) accommodating a medicament container (13), the subassembly (101, 102) comprising:
   a housing (3) having a proximal end (3a) and a distal end (3b),
   an electrical drive unit (82) configured to move a plunger rod (83) of the medicament delivery device (1) in the proximal direction to act on the medicament container (13),
   a battery (85) configured to power the electrical drive unit (82),
   a charging port (87) configured to receive a charger (70) having an abutment surface (72), the charging port (87) having a first electrical connector (88) and being configured to guide a second electrical connector (74) of the charger (70) until the first electrical connector (88) electrically connects to the second electrical connector (74) for charging the battery (85) in a charging event,
   an ejector (50, 150) arranged inside the housing (3) adjacent the charging port (87), the ejector (50, 150) having an ejecting surface (52, 152), wherein the ejector (50, 150) is configured to, during the charging event, be moved into an ejecting state in which the ejecting surface (52, 152) pushes the abutment surface (72) of the charger (70) to thereby eject the charger (70) from the charging port (87) and break the electrical connection between the first and second electrical connectors (88, 74).
2. The subassembly (2, 102) according to clause 1, wherein the ejector is configured to, in the ejecting state, block the charging port from guiding the second electrical connector (74) of the charger (70) to prevent the first electrical connector (88) to electrically connect to the second electrical connector (74).
3. The subassembly (2, 102) according to any one of clauses 1-2, wherein the ejector is configured to be moved into the ejecting state by moving relative to the charging port.
4. The subassembly (2, 102) according to any one of the preceding clauses, wherein the ejector is configured to be moved into the ejecting state by moving along the charging port.
5. The subassembly (2, 102) according to any one of the preceding clauses, wherein the distal end (3b) of the housing (3) comprises a distally facing surface (3c) having an opening (3d), and wherein the charging port (87) is arranged inside the housing (3) at a distal end portion (2b, 102b) of the subassembly (2, 102) and is accessible via the opening (3d).
6. The subassembly (2, 102) according to any one of the preceding clauses, wherein the ejecting surface (52, 152) is a distally facing surface.
7. The subassembly (2, 102) according to clause 6, wherein the ejector (50, 150) is configured to be moved into its ejecting state by being moved distally.
8. The subassembly (2, 102) according to any one of the preceding clauses, wherein the ejector (50, 150) is configured to move from a charging-enabling state in which the ejecting surface (52, 152) is arranged in a non-blocking position enabling the charging port to guide the second electrical connector (74) of the charger (70) such that the electrical connection between the first and second electrical connectors (88, 74) is allowed to the ejecting state.
9. The subassembly (2) according to clause 8, further comprising a spring, or flexible arm (56), configured to bias the ejector (50) to its charging enabling state.
10. The subassembly (2, 102) according to any one of the preceding clauses, further comprising:
   a proximal receiving interface (10) adapted to receive a disposable cassette (4) accommodating the medicament container (13),
   an activation interface (40) and a first switch (90), the activation interface (40) being configured to, upon a disposable cassette installation action, being moved by a part of the disposable cassette (4) to actuate the first switch (90).
11. The subassembly (2) according to clause 10, wherein the ejector (50) extends from a proximal end (50a) to a distal end (50b), and wherein the activation interface (40) comprises a distally facing surface (44a) configured to, upon the disposable cassette installation action, move to abut against and push the proximal end (50a) of the ejector (50) to move the ejector (50) into its ejecting state.
12. The subassembly (2) according to clause 11, wherein the ejector (50) comprises a tubular portion (53) sized and dimensioned to encompass the second electrical connector (74), the tubular portion (53) comprising the ejecting surface (52) and being configured to be arranged between the abutment surface (72) of the charger (70) and the charging port (87) during the charging event.
13. The subassembly (102) according to clause 10, further comprising an electric motor (160) configured to move the ejector (150) into its ejecting state in response to actuation of the first switch (90).
14. The subassembly (102) according to clause 13, wherein the electric motor (160) is a linear motor having a movable piston (162) connected to, or comprising, the ejector (150).
15. The subassembly (2, 102) according to any one of clauses 10-14, further comprising a second switch (92), wherein the activation interface (40) comprises a first distally extending arm (44) extending towards the first switch (90) and second distally extending arm (54) extending towards the second switch (92), and wherein the first distally extending arm (44) is configured to, upon a first movement of the activation interface (40) in the distal direction, reach and actuate the first switch (90), and wherein the second distally extending arm (54) is configured to, upon a second movement of the activation interface (40) in the distal direction, reach and actuate the second switch (92).
16. A subassembly (202) for a medicament delivery device (1) comprising a disposable cassette (4) accommodating a medicament container (13), the subassembly (202) comprising:
   a housing (3) having a proximal end (3a) and a distal end (3b),
   a proximal receiving interface (10) adapted to receive the disposable cassette (4),
   an activation interface (40) and a first switch (90), the activation interface (40) being configured to, upon a disposable cassette installation action, being moved by a part of the disposable cassette (4) to actuate the first switch (90),
   an electrical drive unit (82) configured to move a plunger rod (83) of the medicament delivery device (1) in the proximal direction to act on the medicament container (13),
   a battery (85) configured to power the electrical drive unit (82),
   a charging port (87) configured to receive a charger (70) having an abutment surface (72), the charging port (87) having a first electrical connector (88) and being configured to guide a second electrical connector (74) of the charger (70) until the first electrical connector (88) electrically connects to the second electrical connector (74) for charging the battery (83) in a charging event,
   an electric motor (260) configured to, during the charging event, act on the activation interface (40) to prevent the activation interface (40) to move in the distal direction and thereby preventing actuation of the first switch (90).
17. The subassembly (202) according to clause 16, further comprising a blocking arm (262) configured to be moved by the electric motor (260), wherein the blocking arm (262) is arranged to abut the activation interface (40) during the charging event to prevent the activation interface (40) to move in the distal direction.
18. The subassembly (202) according to clause 17, wherein the electric motor (260) is configured to, upon ceasing of the charging event and removal of the charger (70) from the charging port (87), move the blocking arm (262) distant from the activation interface (40) to enable the activation interface (40) to move in the distal direction and actuating the first switch (90).
19. A medicament delivery device (1) comprising the subassembly (2, 102, 202) according to any one of clauses 1 to 18.

1-15. (canceled)

16. A subassembly for a medicament delivery device accommodating a medicament container, the subassembly comprising:
a housing having a proximal end and a distal end;
an electrical drive unit configured to move a plunger rod of the medicament delivery device in the proximal direction to act on the medicament container;
a battery configured to power the electrical drive unit;
a charging port configured to receive a charger having an abutment surface, the charging port having a first electrical connector and being configured to guide a second electrical connector of the charger until the first electrical connector electrically connects to the second electrical connector for charging the battery in a charging event; and
an ejector arranged inside the housing adjacent to the charging port, the ejector having an ejecting surface, wherein the ejector is configured to, during the charging event, be moved into an ejecting state in which the ejecting surface pushes the abutment surface of the charger to thereby eject the charger from the charging port and break the electrical connection between the first and second electrical connectors.

17. The subassembly according to claim 16, wherein the ejector is configured to, in the ejecting state, block the charging port from guiding the second electrical connector of the charger to prevent the first electrical connector to electrically connect to the second electrical connector.

18. The subassembly according to claim 16, wherein the ejector is configured to be moved into the ejecting state by moving relative to the charging port.

19. The subassembly according to claim 16, wherein the ejector is configured to be moved into the ejecting state by moving along the charging port.

20. The subassembly according to claim 16, wherein the distal end of the housing comprises a distally facing surface having an opening, and wherein the charging port is arranged inside the housing at a distal end portion of the subassembly and is accessible via the opening.

21. The subassembly according to claim 16, wherein the ejecting surface is a distally facing surface.

22. The subassembly according to claim 21, wherein the ejector is configured to be moved into its ejecting state by being moved distally.

23. The subassembly according to claim 16, wherein the ejector is configured to move from a charging-enabling state in which the ejecting surface is arranged in a non-blocking position enabling the charging port to guide the second electrical connector of the charger such that the electrical connection between the first and second electrical connectors is allowed to the ejecting state.

24. The subassembly according to claim 23, further comprising a spring, or flexible arm (56), configured to bias the ejector (50) to its charging enabling state.

25. The subassembly according to claim 16, further comprising:
a proximal receiving interface adapted to receive a disposable cassette accommodating the medicament container; and
an activation interface and a first switch, the activation interface being configured to, upon a disposable cassette installation action, being moved by a part of the disposable cassette to actuate the first switch.

26. The subassembly according to claim 25, wherein the ejector extends from a proximal end to a distal end, and wherein the activation interface comprises a distally facing surface configured to, upon the disposable cassette installation action, move to abut against and push the proximal end of the ejector to move the ejector into its ejecting state.

27. The subassembly according to claim 26, wherein the ejector comprises a tubular portion sized and dimensioned to encompass the second electrical connector, the tubular portion comprising the ejecting surface and being configured to be arranged between the abutment surface of the charger and the charging port during the charging event.

28. The subassembly according to claim 25, further comprising an electric motor configured to move the ejector into its ejecting state in response to actuation of the first switch.

29. The subassembly according to claim 28, wherein the electric motor is a linear motor having a movable piston connected to, or comprising, the ejector.

30. The subassembly according to claim 25, further comprising:
a second switch, wherein the activation interface comprises a first distally extending arm extending towards the first switch and second distally extending arm extending towards the second switch, and wherein the first distally extending arm is configured to, upon a first movement of the activation interface in the distal direction, reach and actuate the first switch, and wherein the second distally extending arm is configured to, upon a second movement of the activation interface in the distal direction, reach and actuate the second switch.
